(19) United States
(12) Patent Application Publication
(10) Pub. No.: US 2020/0185111 A1
(43) Pub. Date: Jun. 11, 2020

(54) MEDICAL INFORMATION PROCESSING SYSTEM AND MEDICAL INFORMATION PROCESSING METHOD

(71) Applicant: OLYMPUS CORPORATION, Tokyo (JP)

(72) Inventors: Hideki TSUJIMOTO, Tokyo (JP); Haruhiko SAKAYORI, Tokyo (JP); Takayuki KURODA, Tokyo (JP)

(73) Assignee: OLYMPUS CORPORATION, Tokyo (JP)

(21) Appl. No.: 16/701,641

(22) Filed: Dec. 3, 2019

Related U.S. Application Data
(63) Continuation of application No. PCT/JP2018/010293, filed on Mar. 15, 2018.

(30) Foreign Application Priority Data
Jun. 6, 2017 (JP) 2017-111921

(51) Int. Cl. G16H 70/20; G16H 30/20; A61B 1/00

(52) U.S. Cl. CPC G16H 70/20; A61B 2505/00; A61B 1/0002; G16H 30/20

(57) ABSTRACT

An implementation information storage stores implementation information of an examination performed by a doctor. A standard range setting unit sets a standard range for implementation information of an examination based on implementation information according to a skilled doctor stored in the implementation information storage. An implementation information acquisition unit acquires implementation information of an examination performed or being performed by a resident doctor. When the implementation information according to the resident doctor falls outside the standard range set by the standard range setting unit, an examination determination unit determines that the examination performed by the resident doctor is not performed properly. A notification processing unit gives notification regarding implementation information of an examination determined not to be performed properly.

### BACKGROUND OF THE INVENTION

### 1. Field of the Invention

The present invention relates to a medical information processing system having a function of determining whether or not a doctor has properly performed an examination.

### 2. Description of the Related Art

Patent Document 1 discloses an examination management system for displaying an overtime examination reason screen for entering the reason for deviation when the actual examination time exceeds an examination target time and registering the reason for an overtime examination in an overtime examination reason table in the database when the reason is entered.

[Patent Document 1] Japanese Patent Application Publication No. 2005-165677

Ideally, inexperienced resident doctors perform endoscopic examinations under the instruction of skilled doctors and create examination reports. However, skilled doctors are busy, and it is practically difficult to always accompany and instruct resident doctors. For this reason, resident doctors often conduct examinations without the attendance of skilled doctors and create examination reports. Therefore, a mechanism is desired that contributes to the improvement of the skill of resident doctors by allowing skilled doctors to efficiently instruct the resident doctors and notifying the resident doctors of the quality of examinations through a system.

### SUMMARY OF THE INVENTION

In this background, a purpose of the present invention is to provide a system having a function of determining whether or not a resident doctor has properly performed an examination.

A medical information processing system according to one embodiment of the present invention includes: an implementation information storage that stores implementation information of an examination performed by a doctor; a standard range setting unit that sets a standard range for implementation information of an examination based on implementation information according to a first doctor stored in the implementation information storage; an implementation information acquisition unit that acquires implementation information of an examination performed or being performed by a second doctor; an examination determination unit that determines that the examination performed by the second doctor is not performed properly when the implementation information according to the second doctor falls outside the standard range that is set by the standard range setting unit; and a notification processing unit that gives notification regarding implementation information of the examination determined not to be performed properly.

Optional combinations of the aforementioned constituting elements and implementations of the invention in the form of methods, apparatuses, systems, recording mediums, and computer programs may also be practiced as additional modes of the present invention.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram illustrating the configuration of a medical information processing system according to an embodiment;

FIG. 2 is a diagram illustrating an example of implementation information acquired by an implementation information acquisition unit;

FIG. 3 is a diagram illustrating an example of examination implementation information stored in an implementation information storage;

FIG. 4 is a diagram illustrating an example of a standard range set by a standard range setting unit;

FIG. 5 is a diagram illustrating an example of a list screen of examination implementation information according to a resident doctor;

FIG. 6 is a diagram illustrating a notification example of information regarding the difference from the standard range;

FIG. 7 is a diagram illustrating a notification example with regard to an examination determined to be improper; and

FIG. 8 is a diagram illustrating an example of report entry screen.

### DETAILED DESCRIPTION OF THE INVENTION

The invention will now be described by reference to the preferred embodiments. This does not intend to limit the scope of the present invention, but to exemplify the invention.

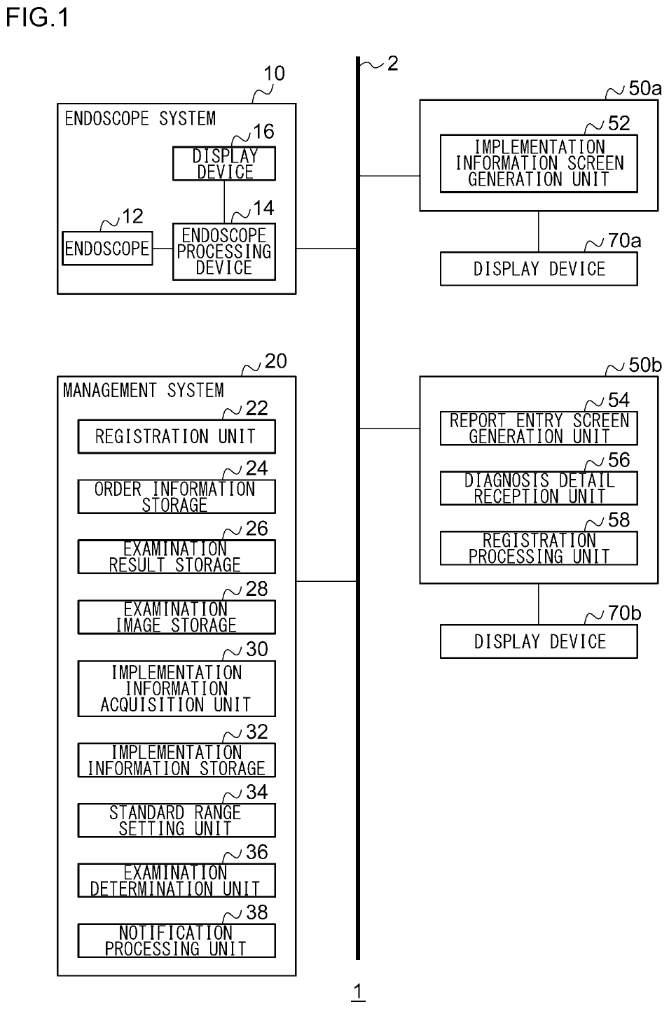

FIG. 1 illustrates the configuration of a medical information processing system 1 according to an embodiment of the present invention. The medical information processing system 1 according to an embodiment is a system for supporting endoscopic examination work in a medical facility. The medical information processing system 1 includes an endoscope system 10, a management system 20, and information processing devices 50a and 50b, which are interconnected by a network 2 such as a LAN (local area network). The information processing devices 50a and 50b (hereinafter, referred to as “information processing devices 50” when the devices are not to be differentiated) are terminal devices such as personal computers and are connected to display devices 70a and 70b to enable output to be displayed on the screen. The information processing devices 50 may be laptop computers integrated with display devices or portable tablets. Further, the information processing devices 50 may be comprised of a combination of a terminal device and a server.

The endoscope system 10 is provided in an endoscopic examination room and includes an endoscope 12, an endoscope processing device 14, and a display device 16. The endoscope processing device 14 is provided with an examination start button and an end button. An examination is started by operating the start button, and the examination is ended by operating the end button. The endoscope processing device 14 transmits examination start information and examination end information to the management system 20 respectively at the timing when the start button is operated and at the timing when the end button is operated.

The endoscope 12 is inserted into the patient's body, and a still image of the inside of the body is captured at the time when the doctor presses a release switch of the endoscope 12. The endoscope 12 is provided with a solid-state imaging device (for example, CCD image sensor or CMOS image sensor) and a signal processing circuit. The solid-state imaging device converts incident light into an electrical signal, and the signal processing circuit performs signal processing such as A/D conversion, noise removal, and the like on image data photoelectric-converted by the solid-state imaging device and outputs the resulting image data to the endoscope processing device 14.

The endoscope processing device 14 controls the entire endoscope system 10 in an integrated manner. One important role of the endoscope processing device 14 is to transmit an examination image captured by the endoscope 12 to the management system 20 and store the examination image in an examination image storage 28. Another important role is to display video acquired by the endoscope 12 on the display device 16 in real time.

In the former role, when the release switch of the endoscope 12 is pressed, the endoscope processing device 14 adds information for identifying the examination (examination ID) and image-capturing time information indicating the image-capturing time to an image captured by the endoscope 12 at least as metadata so as to generate examination image data. The endoscope processing device 14 transmits the examination image data to the examination image storage 28 of the management system 20, and the examination image storage 28 stores the examination image data.

In the endoscope system 10 according to the embodiment, through the endoscope 12 or another means, the performing doctor of the examination can enter to the endoscope processing device 14 information regarding a part into which the endoscope 12 is inserted and also enter the start and end of a predetermined action. Since the doctor operates the endoscope 12, an interface for entry is preferably provided in the endoscope 12 in the form of, e.g., a button or the like. This entry may be performed by a nurse in the examination room in response to a doctor's instruction.

In an upper endoscopic examination, the doctor observes the esophagus, stomach, and duodenum in order. At this time, every time the endoscope 12 is inserted into each part and enters the insertion part to the endoscope processing device 14. The endoscope processing device 14 acquires time for insertion into each part as examination information, and transmits the time for insertion to the implementation information acquisition unit 30 of the management system 20 after the examination is completed. By recording the time when the endoscope 12 is inserted into the esophagus, stomach, and duodenum as examination information, the implementation information acquisition unit 30 can acquire the observation time for each part.

A lower endoscopic examination is an examination in which an endoscope 12 is inserted from the anus to the deepest part of the large intestine (cecum) so as to observe the mucosal surface of the large intestine while moving the endoscope 12 out from the cecum. When the endoscope 12 is inserted up to the cecum, the doctor enters the information indicating that the endoscope 12 has reached the cecum to the endoscope processing device 14. Thereby, the endoscope processing device 14 records the time at which the endoscope 12 is reached the cecum as examination information, and transmits the examination information to the implementation information acquisition unit 30 after the examination is completed.

Further, endoscopic retrograde cholangiopancreatography (ERCP) is an examination in which the bile duct and the pancreatic duct are imaged by inserting an endoscope 12 into the duodenum and inserting a catheter into the pancreatic duct and the bile duct from the distal end of the endoscope 12. When inserting or removing the catheter, the doctor enters the insertion or removal to the endoscope processing device 14. The endoscope processing device 14 records the catheter insertion time and the catheter removal time as examination information and transmits the examination information to the implementation information acquisition unit 30 after the examination is completed. Thereby, the implementation information acquisition unit 30 can acquire the cannulation time (the time during which the catheter has been inserted).

The endoscope processing device 14 transmits examination information entered in each examination to the implementation information acquisition unit 30 of the management system 20 in association with an examination ID. The implementation information acquisition unit 30 acquires examination implementation information from the examination information that is received and stores the examination implementation information in the implementation information storage 32.

The management system 20 includes a registration unit 22, an order information storage 24, an examination result storage 26, an examination image storage 28, an implementation information acquisition unit 30, an implementation information storage 32, a standard range setting unit 34, an examination determination unit 36, and a notification processing unit 38. The order information storage 24 stores order information for an endoscopic examination. The examination result storage 26 stores the examination result of an endoscopic examination, and more specifically stores report information generated by the information processing device 50 in association with examination order information such as patient information, examination type information, and identification information of the doctor who has performed the examination (doctor ID). The report information includes a diagnosis result such as diagnosis details entered by a doctor, a report attachment image selected from examination images that have been captured, a comment regarding the report attachment image, and the like.

The examination image storage 28 stores an examination image captured by the endoscope system 10. The examination image storage 28 may be comprised of a large hard disk drive (HDD) or a flash memory. The examination image storage 28 may be formed as an image database that stores examination images according to a predetermined image standard.

As described above, the implementation information acquisition unit 30 receives examination information from the endoscope processing device 14 and acquires implementation information of the examination from the examination information that is received. The examination information received from the endoscope processing device 14 includes not only information entered by a doctor but also examination start information and examination end information transmitted from the endoscope processing device 14 at the start and end of the examination, respectively. The implementation information acquisition unit 30 defines implementation information of an examination for each examination item. In the embodiment, three types of examination items: upper endoscopic screening examination; lower endoscopic examination; and ERCP, are exemplified. However, all examination items that can possibly performed by resident doctors are practically included.

The configuration shown in FIG. 1 is implemented by hardware such as an arbitrary processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

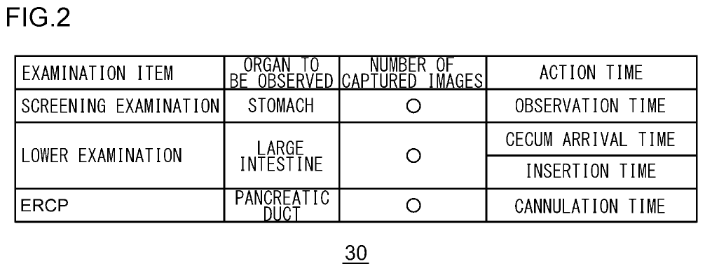

FIG. 2 illustrates an example of implementation information acquired by an implementation information acquisition unit 30. Implementation information acquired by the implementation information acquisition unit 30 is defined for each examination item (organ to be observed). The implementation information acquisition unit 30 stores the implementation information that is acquired in the implementation information storage 32 in association with a doctor ID and an examination item. In the embodiment, it is defined that the implementation information acquisition unit 30 acquires the number of captured examination images as implementation information for all the three types of examination items, and the implementation information acquisition unit 30 may acquire the number of captured images for each examination by checking the number of captured images stored in the examination image storage 28. In FIG. 2, in addition to the number of captured images, the implementation information acquisition unit 30 is defined to acquire the time (action time) required for a predetermined action in an examination as implementation information. The implementation information of an examination may be only either one of the number of captured images or the action time.

For the screening examination, the implementation information acquisition unit 30 acquires the number of captured examination images and stomach observation time as the implementation information. The endoscope processing device 14 transmits the time for insertion into each part to the implementation information acquisition unit 30 as examination information of an upper endoscopic examination. The implementation information acquisition unit 30 obtains the stomach observation time based on the difference between the “time for insertion into the stomach” and the “time for insertion into the duodenum”.

For the lower endoscopic examination, the implementation information acquisition unit 30 acquires the number of captured examination images, cecum arrival time, and endoscope insertion time as the implementation information. The endoscope processing device 14 transmits the examination start time, the time at which the cecum is reached, and the examination end time to the implementation information acquisition unit 30 as examination information of the lower endoscopic examination. The implementation information acquisition unit 30 obtains the cecum arrival time based on the difference between the “time at which the cecum is reached” and the “examination start time”. Further, the implementation information acquisition unit 30 obtains the endoscope insertion time based on the difference between the “examination end time” and the “examination start time”.

For ERCP, the implementation information acquisition unit 30 acquires the number of captured examination images and cannulation time as the implementation information. The endoscope processing device 14 transmits catheter insertion time and catheter removal time to the implementation information acquisition unit 30 as examination information of ERCP. The implementation information acquisition unit 30 obtains the cannulation time based on the difference between the “catheter removal time” and the “catheter insertion time”.

As described above, the implementation information acquisition unit 30 acquires the implementation information of an examination performed by the doctor based on examination information transmitted from the endoscope processing device 14 and stores the acquired information in the implementation information storage 32. The implementation information storage 32 stores examination implementation information in association with a doctor ID.

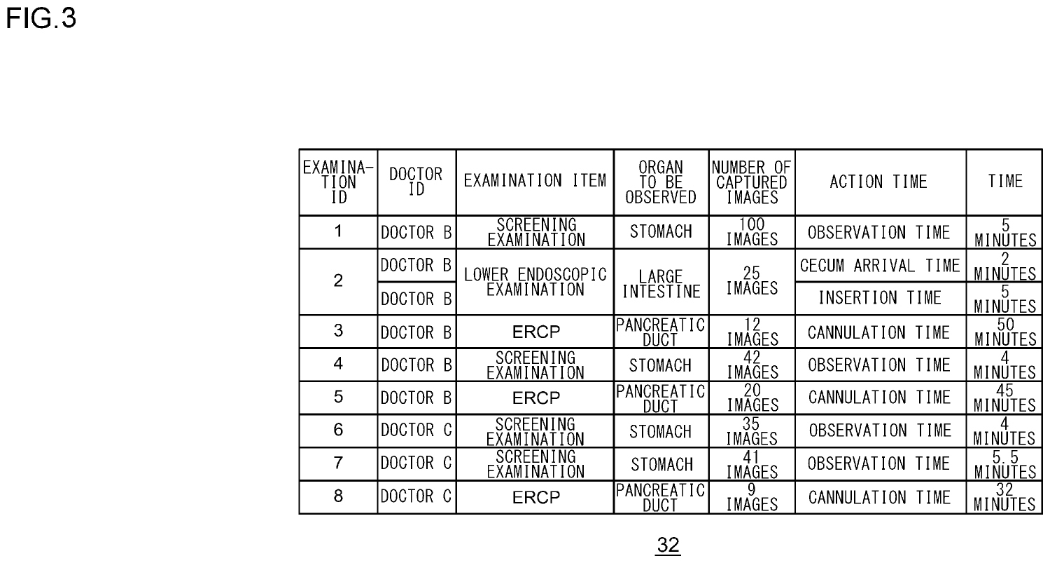

FIG. 3 shows implementation information of examinations performed by doctors B and C stored in the implementation information storage 32. Although FIG. 3 shows only the implementation information of the examinations performed by doctors B and C, the implementation information storage 32 stores the implementation information of examinations performed by all doctors in a medical facility.

In medical facilities, there are inexperienced resident doctors, regular doctors who have gained experience since they were resident doctors, and skilled doctors who have gained more experience since they were regular doctors and have excellent skills. As described above, the implementation information storage 32 stores implementation information of examinations performed by all the doctors including the resident doctors and the skilled doctors. It is assumed that doctors B and C are resident doctors and are to be instructed by skilled doctors.

The medical information processing system 1 according to the embodiment provides a mechanism that allows a skilled doctor to efficiently instruct a resident doctor and a mechanism by which a management system 20 evaluates an examination performed by a resident doctor or an examination that is being performed. In order to realize these mechanisms, the management system 20 creates an index for evaluating the implementation information of an examination performed by a resident doctor. This index is created according to the implementation information of a past examination performed by a skilled doctor such as a supervising doctor.

The standard range setting unit 34 sets a standard range for the implementation information of an examination based on implementation information according to a skilled doctor. The standard range is an index for evaluating the properness of implementation information according to a resident doctor. The standard range setting unit 34 acquires the implementation information of a plurality of examinations performed by a skilled doctor from the implementation information storage 32 for each examination item, and obtains an average value of the implementation information. In the embodiment, setting a standard range using implementation information of a doctor A who is a skilled doctor will be described.

Describing the screening examination, the standard range setting unit 34 acquires the implementation information for a plurality of past screening examinations performed by the doctor A from the implementation information storage 32. In order to obtain an accurate average value, implementation information of, for example, 10 or more examinations is preferably acquired. The implementation information of a screening examination includes the number of captured examination images and the stomach observation time, and the standard range setting unit 34 obtains an average value for the number of captured images and the observation time in a plurality of screening examinations.

When the standard range setting unit 34 obtains the average value of the number of captured images and the average value of the observation time, the standard range setting unit 34 adds a predetermined margin around each average value so as to set a standard range for each average value. That is, when the standard range setting unit 34 obtains the average value of the implementation information of a plurality of examinations, the standard range setting unit 34 sets a standard range so as to include the average value. When the average number of captured examination images in a screening examination is 40, the standard range setting unit 34 may set a standard range with a margin of 20% of the average number around the average number. Therefore, the standard range setting unit 34 sets the standard range of the number of captured images as 32 to 48. In the same manner, when the average observation time in a screening examination is 5 minutes, the standard range setting unit 34 sets the standard range of the observation time to 4 to 6 minutes with a margin of 20% of the average time around the average time. As described above, the standard range setting unit 34 obtains the average value of the implementation information according to a skilled doctor for each examination item and sets a standard range so as to include the average value.

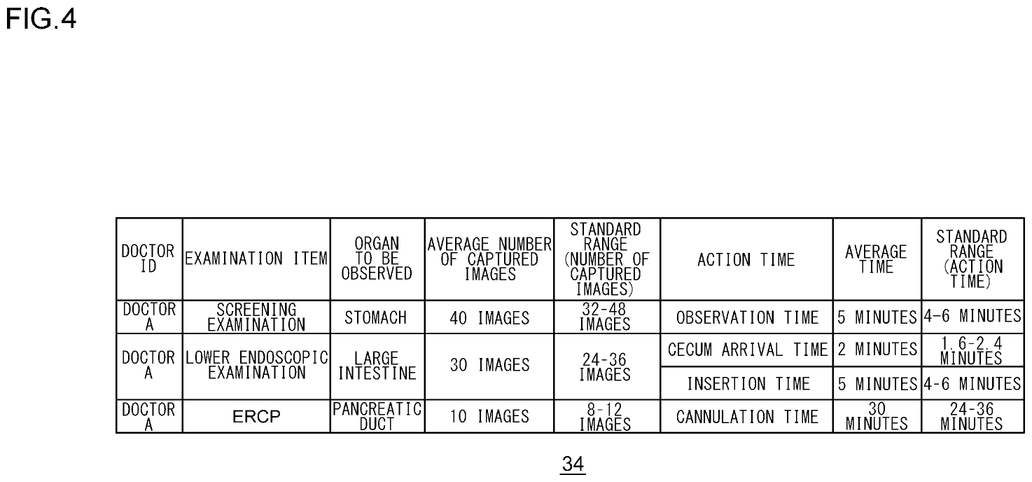

FIG. 4 illustrates an example of a standard range set by the standard range setting unit 34. The standard range setting unit 34 sets a standard range for the number of captured images and the action time for each examination item. The standard range is an index that defines the range of acceptable implementation information. When implementation information of a resident doctor is included in the standard range, there is no problem with the implementation information. However, when the implementation information falls outside the standard range, the implementation information may not be proper.

In the medical information processing system 1, the information processing device 50a is a terminal device operated by a supervising doctor. The supervising doctor may be the same person as the skilled doctor A and has the role of supervising the doctors B and C who are resident doctors at the medical facility. The information processing device 50a includes an implementation information screen generation unit 52 that generates a screen for displaying implementation information of a resident doctor in charge. When the supervising doctor enters a screen generation instruction to the information processing device 50a, the implementation information screen generation unit 52 generates a display screen for the implementation information of the resident doctor and displays the display screen on the display device 70a.

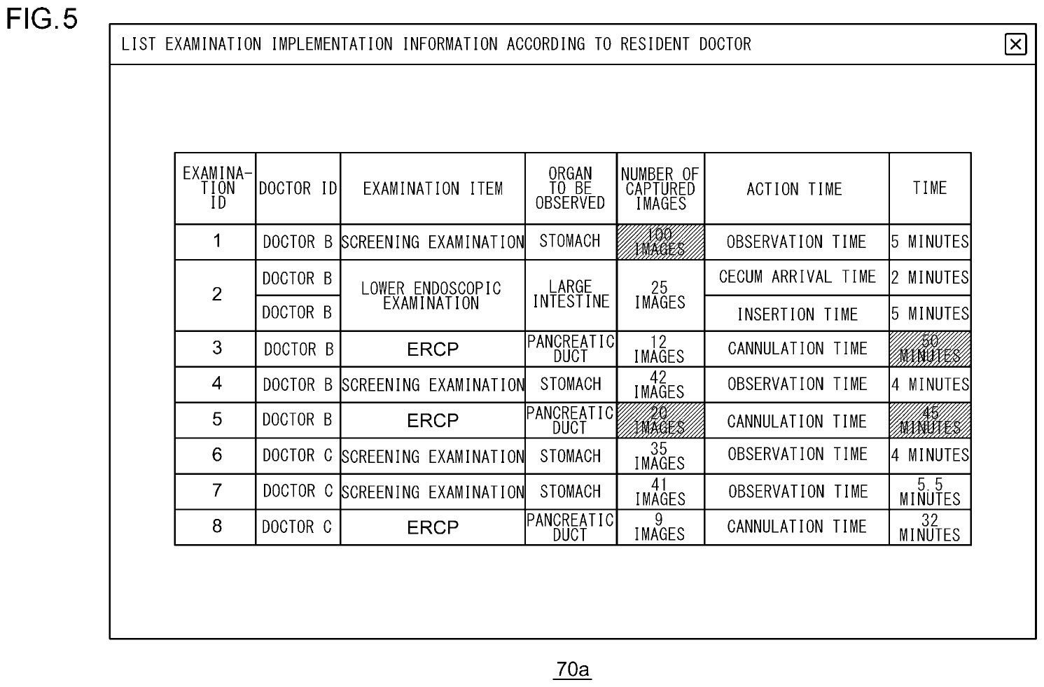

FIG. 5 illustrates an example of a list screen of examination implementation information according to a resident doctor. The examination determination unit 36 determines whether or not implementation information according to a resident doctor is included in a standard range set by the standard range setting unit 34. When the implementation information according to the resident doctor stored in the implementation information storage 32 falls outside of the standard range set by the standard range setting unit 34, the examination determination unit 36 determines that the examination performed by the resident doctor is not performed properly.

The examination implementation information shown in FIG. 5 corresponds to the examination implementation information stored in the implementation information storage 32 shown in FIG. 3. The examination determination unit 36 determines that the number of captured images (100) falls outside the standard range (32 to 48) set by the standard range setting unit 34 in a screening examination performed by a doctor B with an examination ID “1”. The examination determination unit 36 also determines that the cannulation time (50 minutes) falls outside the standard range (24 to 36 minutes) set by the standard range setting unit 34 in a ERCP performed by the doctor B with an examination ID “3”. Further, the examination determination unit 36 determines that the number of captured images (20) falls outside the standard range (8 to 12) and the cannulation time (45 minutes) falls outside the standard range (24 to 36) in an ERCP performed by the doctor B with an examination ID “5”.

Falling outside the standard range indicates that the examination is not performed properly or is likely not performed. The number of captured images of 100 in a screening examination with an examination ID “1” is too large compared to the standard range. Since capturing a large number of images requires a lot of time for diagnosis, the supervising doctor needs to instruct the doctor B to reduce the number of images to be captured in order for an efficient screening examination. Further, the cannulation time is too long in an ERCP with an examination ID an ERCP with an examination ID “3” and an ERCP with an examination ID “5”, and the number of captured images is also slightly too many in the ERCP with the examination ID “5”. Based on these pieces of implementation information, the supervising doctor can determine that the doctor B has a technically unskilled aspect and learns that it is better to call the doctor B to the next ERCP to give the tips of the cannulation.

When the examination determination unit 36 determines that an examination performed by a resident doctor is not properly performed, the notification processing unit 38 gives notification regarding the implementation information of an examination determined to be not performed properly by the examination determination unit 36 on a list screen of examination implementation information shown in FIG. 5. The notification processing unit 38 displays implementation information that falls outside the standard range in bold and displays the display column in a color different from other display columns. This list screen allows the supervising doctor to recognize implementation information that falls outside the standard range at a glance. Further, by displaying the implementation information of properly performed examinations on the list screen, the supervising doctor can learn the strength and weakness of the resident doctor and can efficiently instruct the resident doctor with regard to examinations the resident doctor is poor at.

The notification processing unit 38 may give notification of only the implementation information of an examination that is determined not to be properly performed. At this time, the notification processing unit 38 may give notification of information regarding the difference between the implementation information according to the resident doctor and the average value or the standard range.

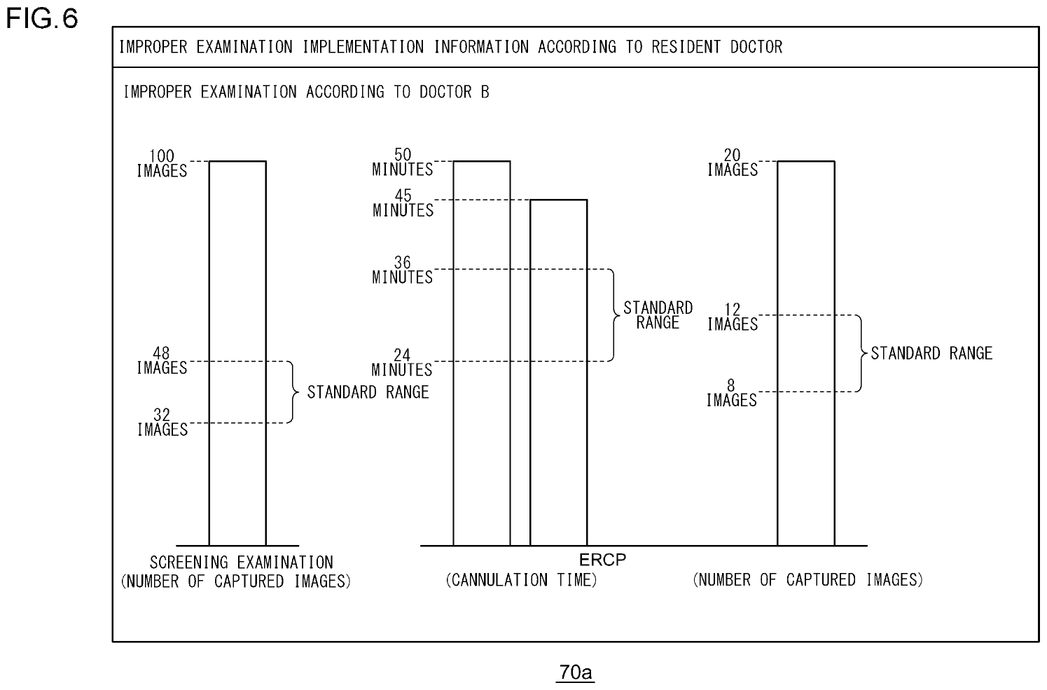

FIG. 6 illustrates a notification example of information regarding the difference from the standard range. In an example shown in FIG. 6, the notification processing unit 38 gives notification of the difference between implementation information and a standard range by indicating the relationship between the standard range and the implementation information in a graph format. The notification processing unit 38 may use other formats to give notification of the difference. Further, the notification processing unit 38 may give notification of the difference from the average value of implementation information of a skilled doctor. The notification processing unit 38 gives notification of the amount of the difference, thereby allowing the supervising doctor to determine the degree of instruction that is necessary. For example, in the example shown in FIG. 6, the amount of deviation from the standard range of the number of captured images in a screening examination is large, and the supervising doctor can learn that instruction should be given immediately for a screening examination.

In the above-described example, the supervising doctor evaluates the implementation information of an examination performed by a resident doctor. Alternatively, a resident doctor may be evaluated by the management system 20 with regard to implementation information during an examination or immediately after the examination is completed.

When the examination end button is operated in the endoscope processing device 14, the endoscope processing device 14 transmits examination information entered by a doctor to the implementation information acquisition unit 30 of the management system 20. The implementation information acquisition unit 30 acquires the implementation information of an examination performed by a resident doctor, and the examination determination unit 36 determines whether or not the implementation information according to the resident doctor is included in a standard range set by the standard range setting unit 34. When the examination determination unit 36 determines that the examination performed by the resident doctor is not proper, the notification processing unit 38 generates a notification detail regarding the implementation information of the examination and transmits the notification detail to the endoscope processing device 14. The endoscope processing device 14 displays the notification detail generated by the notification processing unit 38 on the display device 16.

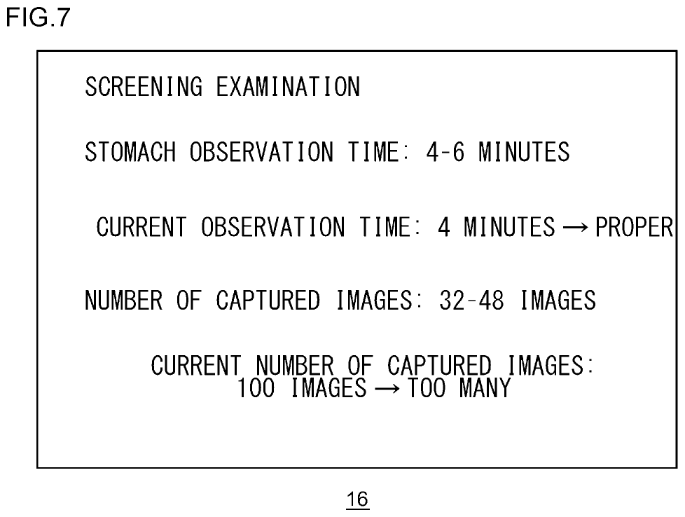

FIG. 7 illustrates a notification example with regard to an examination determined to be improper. In this example, the number of captured images in a screening examination exceeds the standard range, and the endoscope processing device 14 causes the display device 16 to display a notification detail generated by the notification processing unit 38 after the examination is completed. As a result, a resident doctor can immediately learn that the number of captured images is too large in this screening examination.

During an examination, the implementation information acquisition unit 30 may count the number of examination images that are transmitted from the endoscope processing device 14 to the examination image storage 28 and stored. When the number counted by the implementation information acquisition unit 30 exceeds the upper limit of the standard range (48 images), the examination determination unit 36 may determine that the number of captured images is not proper. At this time, the notification processing unit 38 may generate a notification detail indicating that the number of captured images is too large and transmits the notification detail to the endoscope processing device 14. The endoscope processing device 14 may display the notification detail on the display device 16 during the examination.

Next, a mechanism for assisting resident doctors in preparing reports will be described. The information processing device 50b includes a report entry screen generation unit 54, a diagnosis detail reception unit 56, and a registration processing unit 58. When the doctor B who is a resident doctor logs in to the information processing device 50b after an endoscope examination is completed, an application for preparing an examination report is automatically started, and a list of already performed examinations is displayed on a display device 70b. This list of already performed examinations displays examination order information such as a patient name, a patient ID, examination date and time, an examination type, and the like in a list, and the doctor B selects an examination for which a report is to be prepared. When the doctor B selects an examination for which a report is to be prepared from the list of already performed examinations, the report entry screen generation unit 54 generates a report entry screen for entering diagnosis details and displays the report entry screen on the display device 70b.

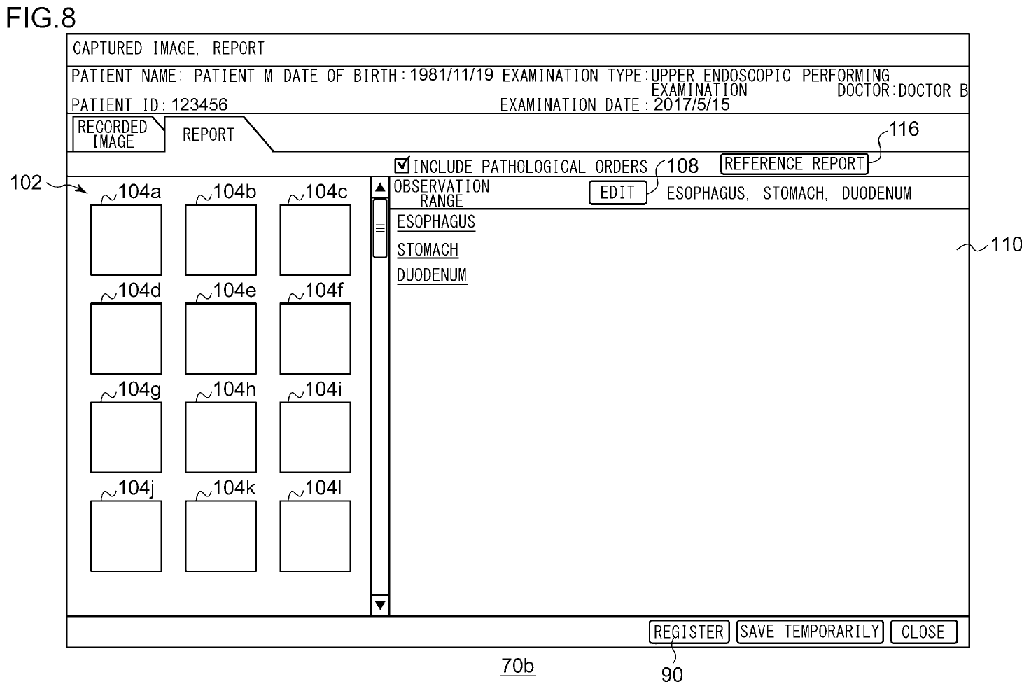

FIG. 8 illustrates an example of a report entry screen. In the upper part of the report entry screen, information on a patient name, a patient ID, the date of birth, an examination type, an examination date, and a performing doctor obtained from the order information storage 24 is displayed. The report entry screen is composed of two areas. An examination image display area 102 for displaying thumbnails of examination images is provided on the left side, and an examination result entry area 110 for a doctor to enter examination results such as diagnosis details is provided on the right side.

The report entry screen generation unit 54 displays thumbnails 104a to 1041 of the examination images acquired from the examination image storage 28 side by side in the examination image display area 102. A scroll bar is provided on the right side of the examination image display area 102, and the doctor B can scroll the scroll bar to observe all the thumbnails of the examination images. The doctor B selects an examination image to be attached to a report while observing the examination images.

The examination result entry area 110 is an area for a doctor to enter an examination result. In the example shown in the figure, an area is provided for entering diagnosis details for “esophagus”, “stomach”, and “duodenum” that are observation ranges in an upper endoscopic examination. When an edit button 108 is operated, selections for an observation range are displayed in a menu window so that the doctor can select an observation range to be diagnosed. The examination result entry area 110 may have a format where a plurality of selections are displayed for examination results such that the doctor enters a diagnosis detail by selecting a check box or may have a free format for free text entry.

On the report entry screen, the doctor enters an examination result including a diagnosis detail in the examination result entry area 110 and selects an examination image to be attached to a report from the examination image display area 102. The diagnosis detail reception unit 56 receives report information including the diagnosis detail entered by the doctor on the report entry screen.

When the report entry screen generation unit 54 generates a report entry screen for an examination that is determined not to be properly performed by the examination determination unit 36, the report entry screen generation unit 54 displays a browse button 116 for browsing diagnosis details entered by skilled doctors on the report entry screen. The browse button 116 is a link button to diagnosis details prepared by skilled doctors for examinations of the same examination item. The doctor B can display an examination report prepared by a skilled doctor on the display device 70b by operating the browse button 116.

In the case of an examination determined to be improper by the examination determination unit 36, it is expected that the doctor B is not only unfamiliar with the examination but also unfamiliar with the preparation of an examination report. Therefore, the report entry screen generation unit 54 includes the browse button 116 in the report entry screen such that the doctor B can easily access the examination reports of the skilled doctors. In the case of an examination determined to be proper by the examination determination unit 36, the report entry screen generation unit 54 may not include the browse button 116 in the report entry screen.

When the doctor B operates a registration button 90 after entering an examination result, the registration processing unit 58 transmits the entered examination result to the management system 20. The examination result includes a diagnosis result entered on the report entry screen, an examination image selected as a report attachment image, and the like. In the management system 20, based on an instruction from the registration processing unit 58, the registration unit 22 stores the diagnosis result and the report attachment image in the examination result storage 26 as report information in association with the doctor ID for identifying the doctor who has entered the diagnosis result and the report attachment image. The report information stored in the examination result storage 26 is printed in a predetermined format and used as an examination report.

Described above is an explanation based on the embodiment of the present invention. The embodiment is intended to be illustrative only, and it will be obvious to those skilled in the art that various modifications to constituting elements and processes could be developed and that such modifications are also within the scope of the present invention.

What is claimed is:

1. A medical information processing system, comprising:
a processor comprising hardware; and
an implementation information storage that stores implementation information including the time required for a predetermined action and the number of captured examination images in an examination performed by a doctor,
wherein the processor is configured to:
set a standard range for implementation information of an examination based on implementation information according to a first doctor stored in the implementation information storage;
acquire implementation information of an examination performed or being performed by a second doctor;
determine that the examination performed by the second doctor is not performed properly when the implementation information according to the second doctor falls outside the standard range that is set; and
give notification regarding implementation information of the examination determined not to be performed properly.

2. The medical information processing system according to claim 1,
wherein the time required for a predetermined action in an examination includes the time required for an endoscope to reach a predetermined part and/or the time required for cannulation.

3. The medical information processing system according to claim 1,
wherein implementation information is defined for each examination item.

4. The medical information processing system according to claim 1,
wherein the processor is configured to:
set the standard range so as to include the average value of implementation information of a plurality of examinations performed by the first doctor.

5. The medical information processing system according to claim 4,
wherein the processor is configured to:
give notification of information regarding the difference between the implementation information according to the second doctor and the average value or the standard range.

6. A medical information processing system, comprising:
a processor comprising hardware; and
an implementation information storage that stores implementation information of an examination performed by a doctor,
wherein the processor is configured to:
set a standard range for implementation information of an examination based on implementation information according to a first doctor stored in the implementation information storage;
acquire implementation information of an examination performed or being performed by a second doctor;
determine that the examination performed by the second doctor is not performed properly when the implementation information according to the second doctor falls outside the standard range that is set;
give notification regarding implementation information of the examination determined not to be performed properly; and
display a browse button for browsing a diagnosis detail entered by the first doctor on an entry screen for entering a diagnosis detail of the examination determined not to be performed properly.

7. The medical information processing system according to claim 6,
wherein implementation information of an examination includes the time required for a predetermined action in an examination and/or the number of captured examination images.

8. The medical information processing system according to claim 7,
wherein the time required for a predetermined action in an examination includes the time required for an endoscope to reach a predetermined part and/or the time required for cannulation.

9. The medical information processing system according to claim 6,
wherein implementation information is defined for each examination item.

10. The medical information processing system according to claim 6,
wherein the processor is configured to:
set the standard range so as to include the average value of implementation information of a plurality of examinations performed by the first doctor.

11. The medical information processing system according to claim 10,
wherein the processor is configured to:
give notification of information regarding the difference between the implementation information according to the second doctor and the average value or the standard range.

12. A medical information processing method comprising:
storing implementation information including the time required for a predetermined action and the number of captured examination images in an examination performed by a doctor in an implementation information storage;
setting a standard range for implementation information of an examination based on implementation information according to a first doctor stored in the implementation information storage;
acquiring implementation information of an examination performed or being performed by a second doctor;
determining that the examination performed by the second doctor is not performed properly when the implementation information according to the second doctor falls outside the standard range that is set; and
giving notification regarding implementation information of an examination determined not to be performed properly.
